(19) United States
(12) Patent Application Publication
(10) Pub. No.: US 2012/0295214 A1
(43) Pub. Date: Nov. 22, 2012

(54) DENTAL AUTO-MIXING METHODS, DEVICES, AND COMPOSITIONS

(76) Inventors: Yizhong Wang, Woodbury, MN (US); Afshin Falsafi, Woodbury, MN (US); Joel D. Oxman, Minneapolis, MN (US); Tiffany T. Ton, Woodbury, MN (US)

(21) Appl. No.: 13/519,805

(22) PCT Filed: Dec. 16, 2010
(86) PCT No.: PCT/US10/60701

§ 371 Date: Jun. 28, 2012

Related U.S. Application Data
(60) Provisional application No. 61/290,538, filed on Dec. 29, 2009.

(51) Int. Cl. A61C 5/00; A61C 7/16; A61C 5/02; A61K 6/083; A61C 5/04

(52) U.S. Cl. 433/24; 523/116; 433/228.1; 433/90; 433/224; 433/217.1

(57) ABSTRACT

A method of dispensing a hardenable dental composition comprising 1) providing a two-part hardenable dental composition, and 2) extruding the composition through a static mixer in fluid communication with a first reservoir containing the first part and a second reservoir containing the second part, wherein a plunger is positioned in each reservoir for simultaneously forcing both parts into the static mixer, extruding the composition through the static mixer, and dispensing the composition, and wherein an extrusion force is applied to the plunger for extruding the composition through the static mixer without the aid of a mechanical advantage provided by an attached or external device. Also disclosed is a method of bonding a prosthetic device to a dental structure using the dispensing method, a device for dispensing the composition using the dispensing method, a kit comprising the device, and the composition used in the dispensing method.

### BACKGROUND

Two-part glass ionomer cements have been in dental use for some time. Such materials are comprised of an ionic polymer component and a reactive glass component, which when mixed together in the presence of water undergo a cement setting reaction. These dental materials provide several desirable attributes including prolonged fluoride release, tolerance to moisture and saliva, good mechanical properties and excellent adhesion to dental hard tissues without pretreatments such as conditioners or adhesives. Powder-liquid, powder-paste, paste-paste, paste-liquid, and liquid-liquid two-part cements have been reported. Traditionally, the two parts have been measured and hand mixed or spatulated; although in one alternative a two-compartment capsule with pre-measured powder and liquid components has been used with vibratory mechanical mixing. Various drawbacks have become evident with these materials and methods, including, for example, mechanical strength variability, varying consistencies, unsatisfactory working or setting times, cost per application, multiple dispensing and mixing steps, mechanical mixing equipment and waste.

More recently, the use of auto mixing delivery systems for two component paste/paste dental materials has addressed some of the above limitations, providing some ease of use, time savings and consistent product performance. In the case of glass ionomer cements, such systems have included a device requiring the combination of a cartridge and a device providing a significant mechanical advantage.

However, there continues to be a growing interest in alternative methods and compositions for delivering glass ionomer cements and related materials in a faster, easier and/or more simplified manner.

### SUMMARY

It has now been found that certain multi-part hardenable glass ionomer dental compositions can be dispensed through a static mixer by applying only hand pressure without the aid of a mechanical advantage provided by an attached or external device. The low force required for dispensing the compositions and small size of a dispensing device that can be used with the compositions allow, among other benefits, direct dispensing of the composition in the mouth.

Accordingly, in one embodiment, there is provided a method of dispensing a hardenable dental composition comprising:
(i) providing a multi-part hardenable dental composition comprising:
   a part (A) in the form of a paste, comprising:
      acid-reactive glass particles and a liquid selected from the group consisting of water, a monomer having at least one ethylenically unsaturated group per monomer molecule, and a combination thereof; and
   a part (B) comprising:
      a water miscible polyacid and a liquid selected from the group consisting of water, a monomer having at least one ethylenically unsaturated group per monomer molecule, and a combination thereof;
   wherein:
      water is included in part (A); part (B); or parts (A) and (B);
      the monomer having at least one ethylenically unsaturated group per monomer molecule is included in part (A); part (B); or parts (A) and (B); and
      at least one component for initiating polymerization of the monomer is included in part (A); part (B); or parts (A) and (B); and
(ii) extruding the composition through a static mixer in fluid communication with a first reservoir containing the part (A) and a second reservoir containing the part (B); wherein a plunger is positioned in each reservoir for simultaneously forcing part (A) and part (B) into the static mixer, extruding the composition through the static mixer, and dispensing the composition; and wherein an extrusion force of less than 40 pound-force (178 newtons) according to Test Method I is applied to the plunger for extruding the composition through the static mixer without the aid of a mechanical advantage provided by an attached or external device.

In another embodiment, there is provided a method of bonding a prosthetic device to a dental structure comprising:
dispensing the hardenable dental composition according to the above method onto a surface of a dental prosthetic device, a surface of a dental structure, or a combination thereof;
positioning the device on the dental structure; and
hardening the dental composition;
wherein the prosthetic device is selected from the group consisting of a crown, bridge, inlay, onlay, post, abutment, veneer, and prosthetic tooth; and
wherein the dental structure is a prepared tooth or an implant.

In another embodiment, there is provided a dental device comprising:
a multi-part hardenable dental composition comprising:
   a part (A) in the form of a paste, comprising:
      acid-reactive glass particles and a liquid selected from the group consisting of water, a monomer having at least one ethylenically unsaturated group per monomer molecule, and a combination thereof; and
   a part (B) comprising:
      a water miscible polyacid and a liquid selected from the group consisting of water, a monomer having at least one ethylenically unsaturated group per monomer molecule, and a combination thereof;
   wherein:
      water is included in part (A); part (B); or parts (A) and (B);
      the monomer having at least one ethylenically unsaturated group per monomer molecule is included in part (A); part (B); or parts (A) and (B); and
      at least one component for initiating polymerization of the monomer is included in part (A); part (B); or parts (A) and (B);
a first reservoir containing the part (A);
a second reservoir containing the part (B);
a static mixer in fluid communication with or which can be connected in fluid communication with the first and second reservoirs; and
a plunger positioned in each reservoir for forcing part (A) and part (B) into the static mixer, extruding the composition through the static mixer, and dispensing the composition;
wherein an extrusion force of less than 40 pound-force (178 newtons) according to Test Method I is required for extruding the composition through the static mixer without the aid of an attached or external device for providing a mechanical advantage.

In another embodiment, there is provided a dental kit comprising the above device and a plurality of static mixers adapted for fluid communication with the first and second reservoirs.

In another embodiment, there is provided a multi-part hardenable dental composition comprising:
a part (A) in the form of a paste, comprising:
   acid-reactive glass particles and a liquid selected from the group consisting of water, a monomer having at least one ethylenically unsaturated group per monomer molecule, and a combination thereof; and
a part (B) comprising:
   a water miscible polyacid and a liquid selected from the group consisting of water, a monomer having at least one ethylenically unsaturated group per monomer molecule, and a combination thereof;
wherein:
   water is included in part (A); part (B); or parts (A) and (B);
   the monomer having at least one ethylenically unsaturated group per monomer molecule is included in part (A); part (B); or parts (A) and (B); and
   at least one component for initiating polymerization of the monomer is included in part (A); part (B); or parts (A) and (B);
wherein the composition can be extruded through a static mixer in fluid communication with a first reservoir containing the part (A) and a second reservoir containing the part (B);
wherein a plunger is positioned in each reservoir for simultaneously forcing part (A) and part (B) into the static mixer and extruding the composition through the static mixer; and
wherein an extrusion force of less than 40 pound-force (178 newtons) according to Test Method I is applied to the plunger for extruding the composition through the static mixer without the aid of a mechanical advantage provided by an attached or external device.

### DEFINITIONS

The term “water soluble” refers to a material, such as a monomer, which is partially or fully water soluble and dissolves in water alone in the amount of at least 5 g per liter of water at 25° C.

The term “water insoluble” refers to a material, such as a monomer, which dissolves in water alone in the amount less than 5 g per liter of water at 25° C.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., the range of viscosity ratios 1:0.06 to 1:13 includes 1:0.06 to 1:13, 1:0.1 to 1:13, 1:0.25 to 1:13, 1:0.5 to 1:13, 1:0.6 to 1:13, 1:1 to 1:13, 1:0.06 to 1:10, 1:0.06 to 1:7.5, 1:0.06 to 1:5, 1:0.06 to 1:3.5, 1:0.06 to 1:1, 1:0.1 to 1:10, 1:0.25 to 1:7.5, 1:0.5 to 1:5, 1:0.6 to 1:3.5, 1:0.75 to 1:2, 1:0.9 to 1:1.1, etc.).

The above summary of the present invention is not intended to describe each disclosed embodiment or every implementation of the present invention. The description that follows more particularly exemplifies illustrative embodiments.

### BRIEF DESCRIPTIONS OF THE FIGURE

FIG. 1 is a perspective view of an assembled dental device for mixing and dispensing a multi-part hardenable dental composition as described herein.

FIG. 2 is a perspective view of a static mixer included in the assembled dental device of FIG. 1.

FIG. 3 is an exploded view in perspective of an alternative dental device for mixing and dispensing a multi-part hardenable dental composition as described herein.

FIG. 4 is a cross-sectional view of the device of FIG. 3 in assembled form showing parts (A) and (B) in separate reservoirs prior to being forced into the static mixer.

### DETAILED DESCRIPTION OF ILLUSTRATIVE EMBODIMENTS OF THE INVENTION

As indicated above, previous methods for auto mixing glass ionomer cements have included a device for providing a mechanical advantage. Examples of such devices include dispensing guns and appliers such as GC FujiCEM Automix and Paste Pak Dispenser (both available from GC Corporation, Japan). This has been found to be undesirable, for example, because of the significant bulk required for the mechanical-advantaging device, making direct dispensing at a dental structure in the mouth difficult and/or impractical. The methods, devices, kits, and compositions disclosed herein allow effective static mixing and dispensing of multi-part hardenable dental compositions using hand pressure without mechanical-advantaging devices. As a result, the practitioner may now conduct auto mixing of multi-part hardenable dental compositions, including glass ionomer cements, using a small sized dispensing device and without hand fatigue or exceptional hand strength.

The methods, devices, compositions, and kits presently provided are applicable to multi-dose and unit-dose applications. In multi-dose applications, a replacement static mixer is used with each successive application of the composition. The kit embodiment, therefore, includes a plurality of static mixers.

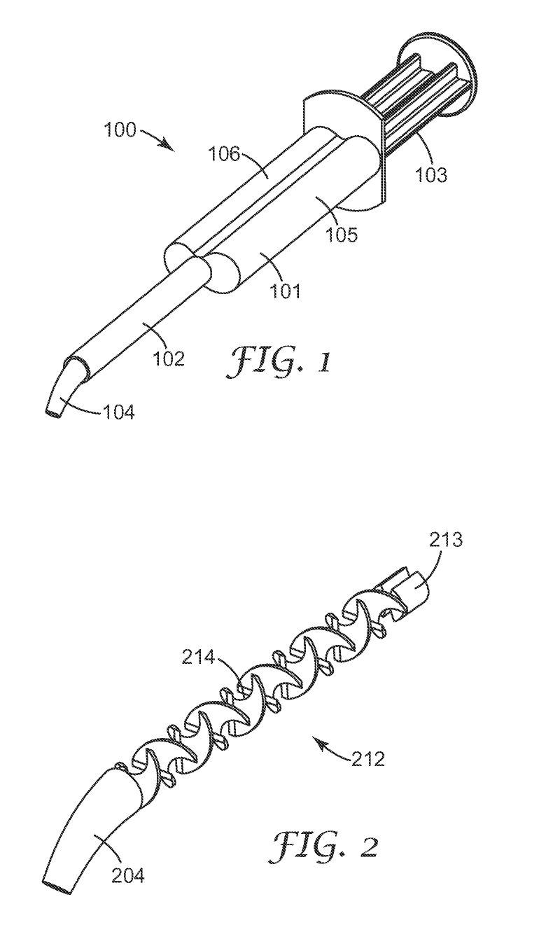

The devices described herein may be provided with a static mixer in fluid communication with the first and second reservoirs or with the static mixer not yet attached, but which can be connected in fluid communication with the first and second reservoirs at an appropriate time. FIG. 1 illustrates one example of an assembled dental device 100 in the form of a double syringe for mixing and dispensing a multi-part hardenable dental composition. Syringe body 101 includes reservoir 105, containing one part of the composition, for example, part (A), and reservoir 106, containing another part of the composition, for example, part (B). Mixing tube 102 contains a static mixer (not shown) and is equipped with optional curved dispensing tip 104. Alternatively, tube 102 may simply taper to a smaller diameter. Mixing tube 102 may be an integral part of syringe body 101, for example, when a unit-dose application is contemplated. Alternatively, mixing tube 102 may be removable and replaceable, for example, when multi-dose applications are to be carried out. Plunger 103 in device 100 is used to force parts (A) and (B) into and through mixing tube 102. As described above, only hand pressure is required to carry this out. It has now been found that an extrusion force of less than 40 pound-force (178 newtons) according to Test Method I described below meets this requirement.

FIG. 2 illustrates static mixer 212 with ten mixing elements 214. In order to achieve adequate and reproducible mixing of parts (A) and (B), a sufficient number of mixing elements arc included. For certain embodiments, including any one of the described method, device, kit, and composition embodiments, preferably the static mixer includes at least 8 mixing elements or at least 10 mixing elements. For certain of these embodiments, the static mixer includes at least 12 mixing elements. While more mixing elements may be used, the number is kept to that which is necessary for adequate and reproducible mixing, so as to prevent unnecessary back pressure resulting from additional, but unnecessary mixing elements. Static mixer 212 is also shown with optional curved dispensing tip 204 and optional closure plug 113, which can function to close outlet openings (not shown) of reservoirs 105 and 106 of device 100 in FIG. 1 to prevent contact between parts (A) and (B) when not in use.

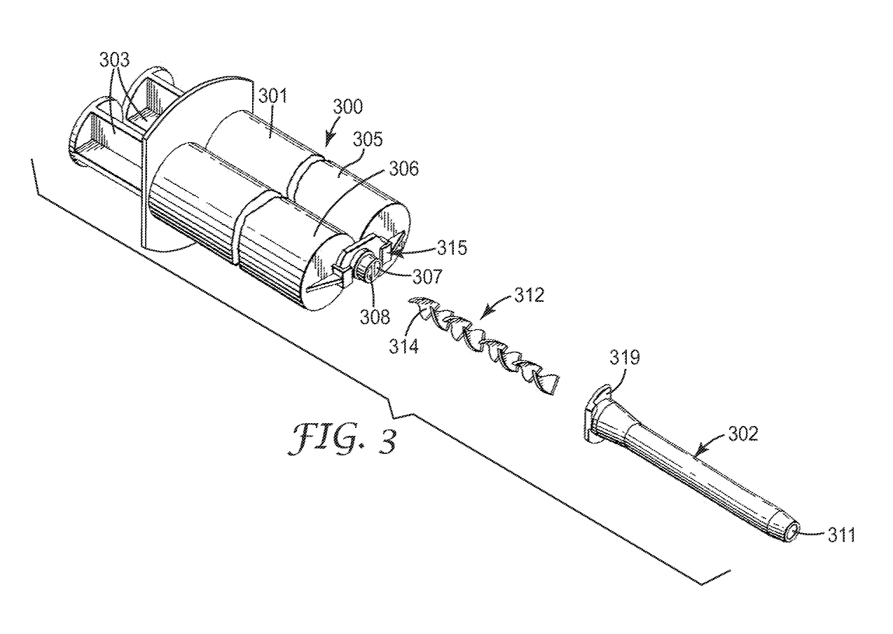

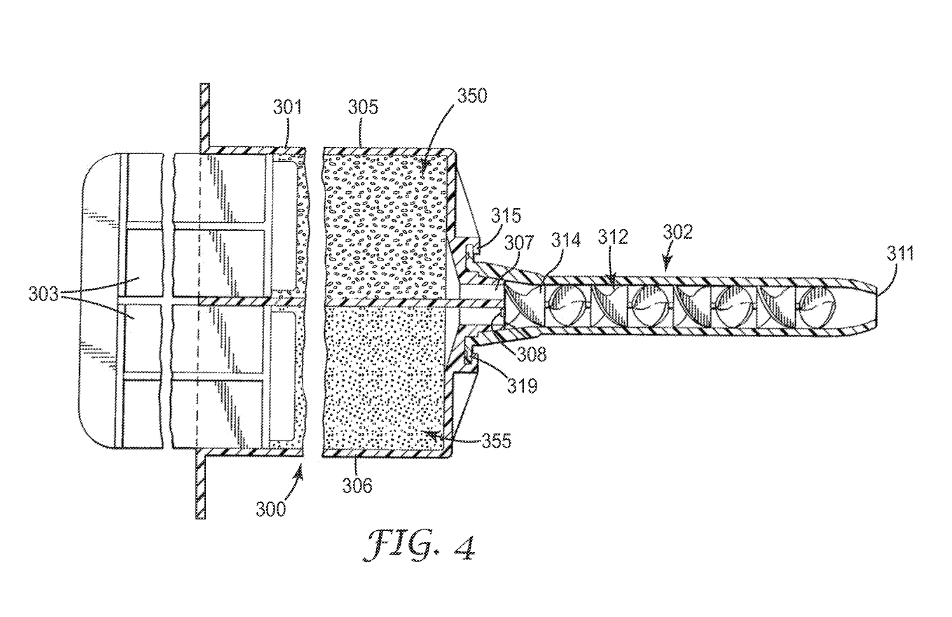

In another example of a device described herein, FIGS. 3 (exploded view in perspective) and 4 (cross-sectional view) illustrate device 300, also in the form of a double syringe, for mixing and dispensing the multi-part hardenable dental composition. Syringe body 301 includes reservoir 305, containing part (A) 350 of the composition, and reservoir 306, containing part (B) 355 of the composition. Mixing tube 302 contains a static mixer 312 with mixing elements 314 and is equipped with outlet 311. Mixing tube 302 is removable and replaceable for multi-dose applications. When mixing tube 302 is installed on syringe body 301, locking ramps 319 of mixing tube 302 are retained by locking tabs 315. Plunger 303 in device 300 is used to force parts (A) 350 and (B) 355 through exit passages 307 and 308 into and through mixing tube 302 with a relatively low force as described above.

Additional examples of specific device constructions, which may be used herein, are described, for example, in U.S. Pat. No. 4,538,920 (Drake) and U.S. Publication No. 2007/016660 A1 (Peuker et al.), the disclosures of which are incorporated by reference in their entirety.

The multi-part hardenable dental compositions described herein advantageously require only a low extrusion force when mixed and dispensed according to the above methods and in the above described device embodiments, while also providing sufficient strength for permanently bonding a prosthetic device to a dental structure. For certain embodiments, including any one of the above method, device, kit, and composition embodiments, when part (A) is mixed with part (B) and the mixture hardened, Shear Bond Strength according to Test Method H (described below) of the resulting hardened cement is greater than 2.0 MPa. For certain of these embodiments, preferably the Shear Bond Strength is greater than 3 MPa, more preferably greater than 4 MPa. These bond strength values refer to bond strengths to either dentin or enamel.

For certain embodiments, each part of the multi-part hardenable dental compositions described herein includes a balance of components for ease of compatibilizing each part with the other during mixing. Accordingly, for certain embodiments, including any one of the above embodiments, part (A) comprises the acid-reactive glass particles, and a water soluble liquid monomer having one ethylenically unsaturated group per monomer molecule; and part (B) comprises the polyacid; a water soluble liquid monomer having one ethylenically unsaturated group per monomer molecule; and water. For certain of these embodiments, preferably at least one of the parts of the multi-part hardenable composition includes a component that provides some cross linking in the composition when hardened. For certain of these embodiments, preferably part B further comprises a liquid monomer having at least two ethylenically unsaturated groups per monomer molecule and having a viscosity less than or equal to the viscosity of Bis-GMA (2,2-bis[4-(2-hydroxy-3-methacryloyloxypropoxy)phenyl]propane, CAS No. 1565-94-2, [H₂C═CH(CH₃)CO₂CH₂CH(OH)CH₂OC₆H₄-4-]₂C(CH₃)₂). For certain of these embodiments, the liquid monomer has a viscosity of at most 50 percent of the viscosity of Bis-GMA.

Each part of the multi-part hardenable dental compositions described herein has a viscosity which is balanced with respect to the other parts of the composition. For certain embodiments, preferably the viscosity of each part is less than 20 fold higher or lower than that of any other part of the composition. For certain embodiments, including any one of the above embodiments, part (A) and part (B) each independently have a viscosity not less than 6 pascal-second (Pa·s) and not greater than 100 Pa·s. For certain of these embodiments, the ratio of part (B) to part (A) viscosity is 1:0.06 to 1:13. For certain of these embodiments, preferably the ratio of part (B) to part (A) viscosity is 1:0.6 to 1:3.5, more preferably 1:0.9 to 1:1.6.

It has been found that the viscosity of part (A) can be controlled for low extrusion force and balanced for good mixing, at least in part, by using a combination of coarse and fine particles of the acid-reactive glass. For example, increasing the relative amount of fine particles of acid-reactive glass, which have an average particle diameter of about 0.2 to about 2 micrometers, increases part (A) viscosity. On the other hand, increasing the relative amount of coarse particles, which have an average particle diameter of greater than about 2 to about 30 micrometers, decreases part (A) viscosity. For certain embodiments, the acid-reactive glass particles are a mixture of coarse particles and fine particles, wherein the fine particles have an average particle diameter of about 0.2 to about 2 micrometers, and the coarse particles have an average particle diameter of greater than about 2 to about 30 micrometers. For certain of these embodiments, the weight ratio of fine to coarse particles is 1:3 to 3:1. For certain of these embodiments, the weight ratio of fine to coarse particles is 1:2 to 2:1. For certain of these embodiments, the coarse particles have an average particle diameter of not more than about 20 micrometers. For certain of these embodiments, the coarse particles have an average particle diameter of 3 to 10 micrometers. For certain of these embodiments, the fine particles have an average particle diameter of 0.5 to 1.5 micrometers.

In order to achieve good mixing and low extrusion force as described above and, at the same time, good strength properties, for certain embodiments, the acid-reactive glass particles are present in part (A) in an amount of about 50 to about 90 weight percent. For certain of these embodiments, the acid-reactive glass particles are present in part (A) at about 65 to about 80 weight percent.

For certain embodiments, part (A) includes water. This provides further control of the viscosity of part (A) and may further increase compatibility with other parts of the composition for good mixing. For certain embodiments, the amount of water in part (A) is about 7 to about 15 percent by weight based upon the total weight of part (A).

For the same reasons, for certain embodiments, part (B) includes water in an amount of about 7 to about 15 percent by weight based upon the total weight of part (B).

Nonreactive fillers may also be included in the compositions described herein to control viscosity as well as for other reasons, such as to achieve a desired appearance, impart desired strength properties, impart radiopacity, and the like. For certain embodiments, including any one of the above embodiments, part (A), part (B), or part (A) and part (B) further include a nonreactive filler in an amount of 1 to 40 weight percent based upon the total weight of the part which includes the nonreactive filler.

Non-reactive fillers may be selected from one or more of any material suitable for incorporation in compositions used for medical applications, such as fillers currently used in dental restorative compositions and the like. The filler preferably has a maximum particle diameter less than about 50 micrometers and an average particle diameter less than about 10 micrometers. When the present compositions are used as a luting cement, the filler is finely divided and has a maximum particle diameter less than about 15 micrometers in order to provide a luting cement with a film thickness in accordance with ISO Standard 3107 of less than about 25 micrometers. The filler can have a unimodal or polymodal (e.g., bimodal) particle size distribution. For certain embodiments which includes a nonreactive filler, the nonreactive filler is selected from the group consisting of inorganic material, crosslinked organic material, and a combination thereof. Suitable crosslinked organic materials are insoluble in the composition, and are optionally filled with inorganic filler. The filler should be non-toxic and suitable for use in the mouth. The filler can be radiopaque, radiolucent or non-radiopaque.

Examples of suitable non-reactive inorganic fillers are naturally-occurring or synthetic materials such as quartz, nitrides (e.g., silicon nitride), glasses derived from, for example, Ce, Sb, Sn, Zr, Sr, Ba and Al, colloidal silica, colloidal zirconia, feldspar, borosilicate glass, kaolin, talc, titania, and zinc glass; low Mohs hardness fillers such as those described in U.S. Pat. No. 4,695,251; and submicron silica particles (e.g., pyrogenic silicas such as the “Aerosil” Series “OX 50”, “130”, “150” and “200” silicas sold by Degussa and “Cab-O-Sil M5” silica sold by Cabot Corp.); metallic powders such as those disclosed in U.S. Pat. No. 5,084,491, especially those disclosed at column 2, lines 52-65; and combinations thereof.

Examples of suitable non-reactive organic filler particles include filled or unfilled pulverized polycarbonates, polyepoxides, and the like. Preferred non-reactive filler particles are quartz, submicron silica and zirconia, and non-vitreous microparticles of the type described in U.S. Pat. No. 4,503,169. Mixtures of these non-reactive fillers are also contemplated, as well as combination fillers made from organic and inorganic materials.

For certain embodiments which include a nonreactive filler, the nonreactive filler is selected from the group consisting of fumed silica, zirconia-silica, quartz, nonpyrogenic silica, and combinations thereof.

The surface of the non-reactive filler particles, in certain embodiments, preferably is treated with a coupling agent in order to enhance the bond between the filler and polymerizable components when the composition is hardened. The use of suitable coupling agents include gamma-methacryloxypropyltrimethoysilane, gamma-mercaptopropyltriethoxysilane, gamma-aminopropyltrimethoxysilane, SILQUEST A-1230 (Momentive Performance Chemicals), and the like.

For certain embodiments which include a nonreactive filler, part (B) includes the nonreactive filler in an amount of about 30 to about 40 weight percent based upon the total weight of part (B). For certain of these embodiments, preferably the nonreactive filler is selected from the group consisting of fumed silica, zirconia-silica, quartz, nonpyrogenic silica, and a combination thereof. For certain of these embodiments, preferably the nonreactive filler is silane treated zirconia-silica.

Part (B) may be in the form of a viscous liquid, a gel, or a paste. The viscous liquids and the gels typically contain relatively lower amounts or no nonreactive filler. The pastes typically include relatively larger amounts of nonreactive filler. For certain embodiments, part (B) is in the form of a paste.

As indicated above, the multi-part hardenable compositions described herein include a liquid monomer having at least one ethylenically unsaturated group per monomer molecule, and in certain embodiments, preferably such monomers are partially or fully water soluble. The liquid monomer having at least one ethylenically unsaturated group per monomer molecule has been found to contribute to the ease of compatibilizing each part with the other during auto mixing and achieving the desired viscosity described above for parts (A) and (B). For certain embodiments, preferably ethylenically unsaturated groups include allyl, vinyl, acrylate, and methacrylate groups. For certain embodiments, such monomers have a relatively low molecular weight and include only one ethylenically unsaturated group per monomer molecule. For certain embodiments, preferably the molecular weight of such monomers is about 100 to about 1000. For certain embodiments, including any one of the above embodiments which includes a water soluble liquid monomer having one ethylenically unsaturated group per monomer molecule in the multi-part hardenable composition, the water soluble liquid monomer is selected from the group consisting of 2-hydroxyethyl (meth)acrylate, glycerol mono(meth)acrylate, sugar methacrylates, and a combination thereof. Ethylenically unsaturated compounds with acid functionality and conforming to the above criteria may also be used. These compounds preferably have an acid functionality selected from an oxyacid of carbon, sulfur, phosphorous, and boron, and may be selected from those described in U.S. Pat. No. 7,156,911, Columns 6-7. The entire disclosure of U.S. Pat. No. 7,156,911 is incorporated by reference herein.

Also as indicated above, the multi-part hardenable compositions described herein, in certain embodiments include a monomer having at least two ethylenically unsaturated groups per monomer molecule which provides some cross linking in the composition when hardened. For certain embodiments, this monomer has a viscosity less than Bis-GMA, more preferably not more than about 50 percent of Bis-GMA For certain embodiments, this monomer is included in part (B). For certain embodiments which include a monomer having at least two ethylenically unsaturated groups per monomer molecule, the monomer having at least two ethylenically unsaturated groups per monomer molecule may be water soluble or water insoluble.

For certain of these embodiments, this monomer does not dissolve appreciable amounts of the polyacid, for example, less than about 5 percent by weight of the polyacid. For certain of these embodiments, the monomer is water insoluble. For certain of these embodiments, the monomer is glycerol dimethacrylate. Alternatively or additionally, a water soluble monomer is used. Suitable water soluble dimethacrylates include various molecular weights of polyethylenglycol (dimeth)acrylates ranging from approximately 400 to 1000 weight average molecular weight. Ethylenically unsaturated compounds with acid functionality having at least two ethylenically unsaturated groups per monomer molecule and conforming to the above criteria may also be used. These compounds preferably have an acid functionality selected from an oxyacid of carbon, sulfur, phosphorous, and boron, and may be selected from those described in U.S. Pat. No. 7,156,911, Columns 6-7.

Suitable water miscible polyacids for part (B) include, but are not limited to, homo- or copolymers of unsaturated mono-, di-, and tricarboxylic acids, for example, homo- or copolymers of acrylic acid, itaconic acid and maleic acid. For certain embodiments, preferably the water miscible polyacids comprise a polymer having sufficient pendent ionic groups to undergo a setting reaction in the presence of a reactive filler and water, and sufficient pendent non-ionically polymerizable groups to enable the resulting mixture to be cured by a redox curing mechanism and/or by exposure to radiant energy.

For certain embodiments, the polyacid is of the Formula I:

B(X)ₘ(Y)ₙ I

wherein B is an organic backbone, each X independently is an ionic group which can undergo a setting reaction in the presence of water and the acid-reactive glass particles, each Y independently is a non-ionically polymerizable group, m is at least 2, and n is at least 1. For certain of these embodiments, X is —COOH and Y is an ethylenically unsaturated group. For certain of these embodiments, the backbone B is an oligomeric or polymeric backbone of carbon-carbon bonds, optionally containing non-interfering substituents such as oxygen, nitrogen or sulfur heteroatoms. The term “non-interfering” refers to substituents or linking groups that do not unduly interfere with either the ionic or the non-ionic polymerization reaction. For certain of these embodiments, preferably B is a hydrocarbon backbone. X and Y groups can be linked to the backbone B directly or by means of any non-interfering linking group, such as substituted or unsubstituted alkylene, alkyleneoxyalkylene, arylene, aryleneoxyalkylene, alkyleneoxyarylene, arylenealkylene, or alkylenearylene groups. Alkylene and arylene refer to the divalent forms of alkyl and aryl, respectively. The linking group may also include linkages such as —OC(═O)—, —C(═O)NH—, —NH—C(═O)O—, —O—, and the like, and combinations thereof, wherein each of these may be used in either direction. For certain of these embodiments, Y is attached to B via an amide linkage. For certain of these embodiments, preferably Y is an acryloyloxy, methacryloyloxy, acrylamido, or methacrylamido group.

The polyacid of Formula I can be prepared according to a variety of synthetic routes, including, but not limited to, (1) reacting n X groups of a polymer of the formula B(X)ₘ₊ₙ with a suitable compound in order to form n pendent Y groups, (2) reacting a polymer of the formula B(X)ₘ at positions other than the X groups with a suitable compound in order to form n pendent Y groups, (3) reacting a polymer of the formula B(Y)ₘ₊ₙ or B(Y)ₙ, either through Y groups or at other positions, with a suitable compound in order to form m pendent X groups and (4) copolymerizing appropriate monomers, e.g., a monomer containing one or more pendent X groups and a monomer containing one or more pendent Y groups. The synthetic route (1) above is preferred. Such groups can be reacted by the use of a “coupling compound”, i.e., a compound containing both a Y group and a reactive group capable of reacting with the polymer through an X group, thereby covalently linking the Y group to the backbone B in a pendent fashion. Suitable coupling compounds are organic compounds, optionally containing non-interfering substituents and/or non-interfering linking groups between the Y group and the reactive group.

Preferred polyacids of Formula I are conveniently prepared by reacting a polyalkenoic acid (e.g., a polymer of formula B(X)ₘ₊ₙ wherein each X is a carboxyl group) with a coupling compound containing both an ethylenically unsaturated group and a group capable of reacting with a carboxylic acid group. The molecular weight of the resultant ionomers is preferably between about 250 and about 500,000, and more preferably between about 1,000 and about 100,000. As referred to herein, “molecular weight” means weight average molecular weight. These polyacids are selected to be water miscible. Suitable polyalkenoic acids for use in preparing the polyacids used herein include those homopolymers and copolymers of unsaturated mono-, di-, and/or tricarboxylic acids commonly used to prepare glass ionomer cements. Representative polyalkenoic acids are described, for example, in U.S. Pat. Nos. 3,655,605; 4,016,124; 4,089,830; 4,143,018; 4,342,677; 4,360,605; and 4,376,835. Preferred polyalkenoic acids are those prepared by the homopolymerization and copolymerization of unsaturated aliphatic carboxylic acids, for example acrylic acid, 2-chloroacrylic acid, 3-chloroacrylic acid, 2-bromoacrylic acid, 3-bromoacrylic acid, methacrylic acid, itaconic acid, maleic acid, glutaconic acid, aconitic acid, citraconic acid, mesaconic acid, fumaric acid and tiglic acid. Suitable monomers that can be copolymerized with the unsaturated aliphatic carboxylic acids include unsaturated aliphatic compounds such as acrylamide, acrylonitrile, vinyl chloride, allyl chloride, vinyl acetate, and 2-hydroxyethyl methacrylate (“HEMA”). Ter- and higher polymers may be used if desired. For certain embodiments, preferably the homopolymers and copolymers of acrylic acid are used. The polyalkenoic acid should be substantially free from unpolymerized monomers and other undesirable components. For certain embodiments, preferably the polyalkenoic acids include polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof.

Polymers of formula B(X)ₘ₊ₙ can be prepared by copolymerizing an appropriate mixture of monomers and/or comonomers. Preferably, such polymers are prepared by free radical polymerization, e.g., in solution, in an emulsion, or interfacially. Such polymers can be reacted with coupling compounds in the presence of appropriate catalysts.

As indicated above, coupling compounds suitable for preparing polyacids for use herein include compounds that contain at least one group capable of reacting with X in order to form a covalent bond, as well as at least one polymerizable ethylenically unsaturated group. When X is carboxyl, a number of groups are capable of reacting with X, including both electrophilic and nucleophilic groups. Examples of such groups include hydroxyl, amino, isocyanato, halo carboxyl, and oxiranyl. Examples of suitable coupling compounds include, but are not limited to, acryloyl chloride, methacryloyl chloride, vinyl azalactone, allylisocyanate, 2-hydroxyethyl methacrylate, 2-aminoethyl methacrylate, and 2-isocyanatoethyl methacrylate. Other examples of suitable coupling compounds include those described in U.S. Pat. Nos. 4,035,321 and 5,814,682, the disclosures of which are hereby incorporated by reference.

For certain embodiments, including any one of the above embodiments, the polyacid is selected from the group consisting of the reaction product of a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof with a coupling compound selected from the group consisting of acryloyl chloride, methacryloyl chloride, vinyl azalactone, allylisocyanate, 2-hydroxyethyl methacrylate, 2-aminoethyl methacrylate, and 2-isocyanatoethyl methacrylate.

Suitable acid-reactive glass includes ion-leachable glasses, e.g., as described in U.S. Pat. Nos. 3,655,605; 3,814,717; 4,143,018; 4,209,434; 4,360,605 and 4,376,835. For certain embodiments, the acid-reactive glass is preferably selected from borate glasses, phosphate glasses and fluoroaluminosilicate glasses. For certain embodiments, the acid-reactive glass is fluoroaluminosilicate (FAS) glass. Suitable acid-reactive glasses are also available from a variety of commercial sources familiar to those skilled in the art. For example, suitable acid-reactive glasses can be obtained from a number of commercially available glass ionomer cements, such as “GC Fuji LC” (GC Corporation) cement and “Kerr XR” (Kerr Corporation) ionomer cement. Mixtures of acid-reactive glasses can be used if desired.

The acid-reactive glass particles may also be subjected to a surface treatment. Suitable surface treatments include acid washing, treatment with phosphates, treatment with chelating agents such as tartaric acid, treatment with a silane or silanol coupling agent. For certain embodiments, preferably the acid-reactive glass particles are silanol treated fluoroaluminosilicate glass particles, as described in U.S. Pat. No. 5,332,429, the disclosure of which is incorporated by reference herein.

When mixing the parts of the multi-part composition, it has been found that better mixing occurs when the parts are in a volume ratio approaching or at about 1:1, as compared with using relatively larger differences in the volumes. This results in better properties of the composition when hardened, for example, higher shear bond strength and/or diametral tensile strength (DTS). Moreover, mixing parts in volumes that are very different from each other increases the possibility of introducing error into the amounts of the components, which would adversely affect properties. Accordingly, for certain embodiments, including any one of the above embodiments, the part (A) and the part (B) are in a volume ratio of 1.2:1 to 1:1.2.

As indicated above, an extrusion force of less than 40 pound-force (178 newtons) according to Test Method I applied to the plunger for extruding the present composition through the static mixer can now be carried out without the aid of a mechanical advantage provided by an attached or external device. Extrusion forces considerably lower than 178 newtons have now been achieved. For certain embodiments, including any one of the above embodiments, the force is less than 35 pound-force (156 newtons) less than 30 pound-force (133 newtons). For certain of these embodiments, the force is less than 20 pound-force (89 newtons). For certain of these embodiments, the force is 10 to 15 pound-force (44 to 67 newtons). It is noted that stiction can make dispensing the composition with an even lower extrusion force, such as an extrusion force of 5 pound-force or less, undesirable. This is because the plunger may momentarily stick, and overcoming this stiction may require less force than that required to dispense the composition, resulting in an uncontrolled amount of composition being dispensed.

The multi-part hardenable dental composition used in the embodiments described herein include at least one component for initiating polymerization of the monomers in the composition and thereby further harden and strengthen the composition to a level greater than that provided by the ionic setting reaction, which occurs between the acid-reactive glass particles and the polyacid. For certain embodiments, the multi-part hardenable dental composition can undergo hardening by heat or light activated polymerization or redox polymerization. For certain of these embodiments, the multi-part hardenable dental composition can undergo hardening by photopolymerization or redox polymerization.

Redox polymerization is provided by separately incorporating an oxidizing agent and a reducing agent as a redox catalyst system into the dental composition for curing via a redox reaction. Various redox systems and their use in ionomer cements are described in U.S. Pat. No. 5,154,762, the disclosure of which is incorporated herein by reference. A metal complexed ascorbic acid is a preferred reducing agent that provides cure with excellent color stability. This reducing agent and redox system is more fully described in U.S. Pat. No. 5,501,727, the disclosure of which is incorporated herein by reference. The oxidizing agent should react with or otherwise cooperate with the reducing agent to produce free radicals capable of initiating polymerization of the ethylenically unsaturated groups. The amount for each of the reducing agent and the oxidizing agent is about 0.01 to about 10%, or in some embodiments, about 0.02 to about 5%, based on the total weight (including water) of the unset composition.

The oxidizing agent and the reducing agent preferably are sufficiently shelf stable and free of undesirable coloration to permit their storage and use under typical dental conditions. The oxidizing agent and the reducing agent are sufficiently soluble and present in an amount sufficient to permit an adequate free radical reaction rate. This can be evaluated by combining all of the ingredients of the cement except for the filler under safelight conditions and observing whether or not a hardened mass is obtained.

Suitable oxidizing agents include persulfates such as sodium, potassium, ammonium and alkyl ammonium persulfates, benzoyl peroxide, hydroperoxides such as cumene hydroperoxide, tert-butyl hydroperoxide, tert-amyl hydroperoxide and 2,5-dihydroperoxy-2,5-dimethylhexane, salts of cobalt (III) and iron (III), hydroxylamine, perboric acid and its salts, salts of a permanganate anion, and combinations thereof. Hydrogen peroxide can also be used, although it may, in some instances, interfere with the photoinitiator, if one is present. The oxidizing agent may optionally be provided in an encapsulated form as described in U.S. Pat. No. 5,154,762.

Reducing agents include ascorbic acid, metal complexed ascorbic acid, aromatic amines such as dimethylaminophenethanol and dihydroxyethyl-p-toludine, cobalt (II) chloride, ferrous chloride, ferrous sulfate, hydrazine, hydroxylamine, oxalic acid, thiourea, alkyl thioureas and salts of a dithionite, 1-allyl-2-thiourea, thiosulfate, aromatic sulfinic acid salts such as benzene sulfinic salts and p-toluenesulfinic salts, sulfite anion and a combination thereof. Ascorbic acid and aromatic tertiary amines are preferred reducing agents. For certain embodiments, a secondary ionic salt may be used to enhance stability of the system, such as described in U.S. Pat. No. 6,982,288.

The ionomer cement systems of the invention may optionally contain one or more suitable initiators that act as a source of free radicals when activated by heat or light. Such initiators can be used alone or in combination with one or more accelerators and/or sensitizers. The initiator should be capable of promoting free radical polymerization and/or crosslinking of the ethylenically unsaturated moiety on exposure to light of a suitable wavelength and intensity. The initiator preferably is also sufficiently shelf stable and free of undesirable coloration to permit its storage and use under typical dental conditions. Visible light photoinitiators are preferred. The photoinitiator preferably is partially or fully soluble in the combined liquid components of the composition parts (A and B).

Free radical-generating photoinitiators may be used alone, but in certain embodiments, preferably are used in combination with a photosensitizer and/or an accelerator. Such initiators can generate free radicals for addition polymerization upon exposure to light energy having a wavelength between 200 and 800 nanometers.

Suitable photoinitiators (i.e., photoinitiator systems that include one or more compounds) include binary and ternary photoinitiators. In one example, a ternary photoinitiator may include an iodonium salt, a photosensitizer, and an electron donor compound as described in U.S. Pat. No. 5,545,676 (Palazzotto et al.). Examples of iodonium salts include diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Examples of photosensitizers include monoketones and diketones that absorb some light within a range of about 400 nanometers to 520 nanometers, preferably 450 to 500 nanometers. Preferred are alpha diketones that absorb light within these ranges. Examples of such photosensitizers include camphoroquinone, benzil, furil, 3,3,6,6-tetramethylcyclohexanedione, phenanthraquinone, 1-phenyl-1,2-propanedione, and other 1-aryl-1 -alkyl-1,2-ethanediones, and cyclic alpha diketones. Most preferred is camphoroquinone. Preferred electron donor compounds include substituted amines, e.g., ethyl dimethylaminobenzoate.

The photoinitiator, when utilized, should be present in an amount sufficient to provide the desired rate of polymerization. This amount will be dependent in part on the light source, the thickness of the layer to be exposed to radiant energy, and the extinction coefficient of the photoinitiator. Typically, the photoinitiator components will be present at a total weight of about 0.01 to about 5%, more preferably from about 0.1 to about 5%, based on the total weight of the composition.

Additional components, which are suitable for use in the oral environment, may optionally be used in the multi-part hardenable compositions described herein. In one example, such components include solvents, cosolvents (e.g., alcohols) or diluents. In another example, indicators, dyes, pigments, inhibitors, accelerators, viscosity modifiers, wetting agents, tartaric acid, chelating agents, surfactants, buffering agents, stabilizers (including free-radical stabilizers), submicron silica particles, additives that impart fluorescence and/or opalescence, modifying agents that prolonged working time, and other materials that will be apparent to those skilled in the art may be used. Additionally, medicaments or other therapeutic substances can be optionally added to the compositions. Examples include whitening agents, breath fresheners, flavorants, fragrances, anticaries agents (e.g., xylitol), fluoride sources, remineralizing agents (e.g., calcium phosphate compounds), enzymes, anesthetics, clotting agents, acid neutralizers, chemotherapeutic agents, immune response modifiers, thixotropes, polyols, anti-inflammatory agents, antimicrobial agents, antifungal agents, agents for treating xerostomia, desensitizers, and the like of the type which may be used in dental compositions. Combinations of any of the above additives may also be used in the compositions described herein. The selection and amount of any one such additive can be determined by one of skill in the art according to the desired result.

Modifying agents which may prolong the time between the beginning of the setting reaction in a restoration and the time sufficient hardening has occurred to allow subsequent clinical procedures to be performed on the surface of the restoration include, e.g., alkanolamines such as ethanolamine and triethanolamine, and mono-, di-, and tri-sodium hydrogenphosphates. Modifying agents can be added to either part A or part B. When used, they are present at a concentration between about 0.1 to 10 percent by weight, based on the total composition weight.

Certain stabilizers provide color stability. Such stabilizers include oxalic acid, sodium metabisulfite, sodium bisulfite, sodium thoisulfate, metaphosphoric acid, and combinations thereof.

Free radical stabilizers can be used with a photoinitiator to prevent premature polymerization or to adjust the working time in free radically initiated compositions. Suitable examples of free radical stabilizers include, e.g., butylated hydroxytoluene (BHT) and methyl ethyl hydroquinone (MEHQ).

Submicron silica particles may be used to improve the handling properties. Suitable silica particles include pyrogenic silicas such as AEROSIL series OX 50, 130, 150, 200, and R-812S, available from Degussa Corp., and CAB-O-SIL M5 silica available from Cabot Corporation.

Viscosity modifiers include thickening agents. Suitable thickening agents include hydroxypropyl cellulose, hydroxymethyl cellulose, carboxymethylcellulose and its various salts such as sodium, and combinations thereof.

The methods, devices, and compositions described herein are well suited for a number of dental applications, such as, for example, a luting cement used to anchor or hold a prosthetic device (e.g., crown, bridge, inlay, onlay, post, abutment, veneer, prosthetic tooth, and the like) in place in the mouth; a restorative or filler material used, for example, for filling a cavity; a thin film used, for example, as a liner on dentin and enamel or a sealant or sealing material on enamel; an orthodontic bracket adhesive; a band cement; and the like. For certain embodiments, the multi-part hardenable dental composition is selected from the group consisting of a liner material, a luting material, a restorative material, an endodontic material, and a sealing material. For certain embodiments, including any one of the above embodiments, the multi-part hardenable dental composition is an orthodontic bracket adhesive material or band cement.

Objects and advantages of this invention are further illustrated by the following examples, but the particular materials and amounts thereof recited in these examples, as well as other conditions and details, should not be construed to unduly limit this invention.

### EXAMPLES

Abbreviations, Descriptions, and Sources of Materials

| | Description and Source of Material (Unless otherwise |
|---|---|
| Abbreviation | indicated, available from Sigma-Aldrich, St. Louis, MO.) |
| ATU | allyl thiourea (Sigma Aldrich, St. Louis, MO) |
| Bis-GMA | 2,2-bis[4-(2-hydroxy-3- |
| | methacryloyloxypropoxy)phenyl]propane |
| | (CAS No. 1565-94-2) (ESSTECH CORP, Essington PA) |
| BHT | 2,6-di-tert-butyl-4-methylphenol (Sigma Aldrich, St. |
| | Louis, MO) |
| DMAPE | 4-dimethylaminophenethyl alcohol (Sigma Aldrich, St. |
| | Louis, MO) |
| DI W | deionized water |
| Zr-Si Filler | silane treated zirconia-silica filler prepared as described |
| | in U.S. Pat. No. 6,818,682 at col. 11, line 41 through col. |
| | 12, line 10. (The entire contents of 6,818,682 |
| | incorporated by reference). |
| GDMA | glycerol dimethacrylate (Sigma Aldrich, St. Louis, MO) |
| HEMA | 2-hydroxycthyl methacrylate (Sigma Aldrich, St. Louis, |
| | MO) |
| Micro TiO₂ | titanium dioxide (Degussa, Parsippany, NJ) |
| PP | potassium persulfate (Sigma Aldrich, St. Louis, MO) |
| R812S | fumed silica (Degussa, Parsippany, NJ) |
| ST Fine Glass | Fine FAS glass with silane treatment having a particle |
| | size of about 1 micrometer and treated as described in |
| | U.S. Pat. No. 7,173,074, column 13 (Mo-Sci Corp., |
| | Columbia, MO) |
| ST Coarse Glass | Coarse FAS glass with silane treatment having a particle |
| | size of about 3 micrometers and treated as described in |
| | U.S. Pat. No. 7,173,074, column 13 (Mo-Sci Corp., |
| | Columbia, MO) |
| SW | DI water saturated with K₂SO₄ and NaH₂PO₄ |
| VBCP | Reaction product of 2-isocyanatoethyl methacrylate and |
| | a copolymer of acrylic acid and itaconic acid prepared as |
| | described in U.S. Pat. No. 5,130,347, Example 11 |

### Dispensing Equipment

Auto mixing was carried out using a MIXPAC syringe (Sulzer Chemtech, Switzerland) with a medium auto mixing tip. The MIXPAC syringe is 5 ml syringe with dual barrels (1:1 volume ratio) for multi-dose applications. The part numbers of the syringe parts and mixing tip were as follows:

| | |
|---|---|
| SDL X05-01-52 | 5 ml double syringe, black |
| | (each barrel having volume of 5 mL) |
| PPD X05-01-10SI | 5 ml piston with silicone o-ring in each barrel |
| PLH X05-46 | 5 ml plunger |
| ML 2.5-12-SB | 12-clement mixing tip (medium) |
| VL 002-S1 | Cap for protecting the contents of the syringe |
| | during storage |

### Test Method I—Extrusion Force

Extrusion force was tested using an (Instron 1123, Instron Corp. Canton, Mass.) with a crosshead speed at 100 mm/min on the above described 5 ml MIXPAC syringe with the medium mixing tip. The MIXPAC syringe with a medium mixing tip on one end and a plunger inserted in the other end was inserted into a hole on a sample holder, so the Mixpac was held steady. As the plunger was pushed into the syringe, the peak force (extrusion force) required while pushing the plunger a distance of 14 mm into the syringe was measured in unit of pound-force (lb-f).

### Test Method II—Shear Bond Strength

Shear bond strength to enamel or dentin for a given test sample was evaluated by the following procedure.

### Preparation of Teeth:

Bovine incisal teeth, free of soft tissue, were embedded in circular acrylic disks. The embedded teeth were stored in water in a refrigerator prior to use. In preparation for adhesive testing, the embedded teeth were ground to expose a flat enamel or dentin surface using 120-grit sandpaper mounted on a lapidary wheel. Further grinding and polishing of the tooth surface was done using 320-grit sandpaper on the lapidary wheel. The teeth were continuously rinsed with water during the grinding process. The polished teeth were stored in deionized water and used for testing within 2 hours after polishing. The teeth were allowed to warm in a 36° C. oven to between room temperature (23° C.) and 36° C. before use.

### Teeth Bonding:

Prior to bonding, excess moisture was removed from the teeth with a damp towel. A 2.5-mm thick Teflon mold with a hole approximately 4.7 mm in diameter was used to produced a testing button. Place the curved end of the smaller diameter half of the gelatin capsule through Teflon mold hole level with the bottom of the mold. Then with a razor blade, cut off the capsule level with the mold, thus making a sleeve. The Teflon mold with gelatin sleeve was clamped on the embedded tooth so that the hole in the mold is directly over the polished tooth. Then cement was directly extruded from the syringe through the static mixing tip (described above) into the hole and pressed with a spatula to make it the same level as the mold. The excess cement was removed with spatula. Then the bonded tooth was placed in 37° C./95% RH chamber for 20 minutes to cure the cement. The clamp was removed, and the bonded tooth with the mold put into 37° C. water for 24 hr before testing the bonding strength.

### Shear Bond Strength Testing:

Prior to do the testing, the Teflon mold was removed. The shear bond strength of a cured test example was evaluated by mounting the assembly (described above) in a holder clamped in the jaws of an INSTRON testing machine (Instron 1123, Instron Corp. Canton, Mass.) with the polished tooth surface oriented parallel to the direction of pull. A loop of orthodontic wire (0.44-mm diameter) was placed around the button (molded cured cement) adjacent to the polished tooth surface. The ends of the orthodontic wire were clamped in the pulling jaw of the INSTRON apparatus and pulled at a crosshead speed of 2 mm/min, thereby placing the adhesive bond in shear stress. The force in kilograms (kg) at which the bond failed was recorded, and this number was converted to a force per unit area (units of kg/cm² or MPa) using the known surface area of the button. Each reported value of adhesion to enamel or adhesion to dentin represents the average of 5 replicates.

### Test Methods III—Diametral Tensile Strength (DTS)

Briefly, samples were prepared from an auto mixing delivery system by extruding pastes into 4 mm glass tubes or, for comparison, from hand mixed pastes injected into 4 mm glass tubes, curing at room temperature at 40 PSI pressure for 20 minutes, then curing in 37° C. and 97% relative humidity chamber for 1 hr, then stabilizing in 37° C. DI water for 24 hrs before sample cutting using a diamond saw and testing on Instron 4505 following DTS testing methods. The paste A to paste B volume ratio was 1:1 for all formulations.

Auto mixing cement sample was made by direct extrusion of 1:1 volume ratio paste A and paste B through medium auto mixing tip (as described above) into the glass tube.

For comparison to hand mixing, in a controlled environment of 24° C. and 50% relative humidity, a hand mixed cement sample was made by spatulating 3 g of 1:1 volume ratio of paste A and paste B for 30 seconds.

Diametral tensile strength samples were made by first injecting a mixed paste sample into a glass tube having a 4 mm inner diameter. The ends of the glass tube were plugged with silicone plugs. The filled tubes were subjected to 0.275 megapascal (MPa) pressure for 5 minutes. Thereafter, the tube was placed in a humidity chamber set at 97% relative humidity and 37° C. for 1 hr. From the humidity chamber, the tube was moved into 37° C. deionized water for 24 hours.

Seven such cured samples were cut to a length of 2 mm. Diametral tensile strength was determined according to ISO Standard 7489 using an INSTRON universal tester (Instron Corp., Canton, Mass.) operated at a crosshead speed of 1 millimeter per minute (mm/min).

### Test Method IV—Viscosity

Rheological properties were measured on TA instrument AR G2 at room temperature with simple shear mood. Viscosities of different pastes at shear rate 20 s⁻¹ were used for demonstration of balanced viscosities of different pastes

### Preparation of Pastes

Paste A was prepared by adding HEMA, DI water, ATU, and DMAPE in a mixing cup, and speed mixing on a Speed Mixer (from FlackTek Inc, Landrum, S.C.) to from a clear solution. The remaining components were then added according to the formulation, followed by speed mixing at 300 rpm for 2 minutes. Paste mixing uniformity was checked, and, if necessary, mixing was continued at the same rpm until a uniform paste A was formed.

Paste B was prepared by adding HEMA, GDMA, and the saturated potassium salt solution into a mixing cup, and speed mixing to forma a clear solution. BHT and VITREBOND were then added and mixing continued using the speed mixer at 3000 rpm to form a clear solution. The remaining components were then added according to the formulation, followed by speed mixing at 3000 rpm by 2 minutes. Paste mixing uniformity was checked, and, if necessary, mixing was continued at the same rpm until a uniform paste B was formed.

### Example 1-12

Pastes A were prepared as described above, and the resulting paste compositions are shown in Table 1. The viscosity of each of the resulting paste compositions was measured according to Test Method IV, and the results are shown in Table 2.

TABLE 1
Compositions of Pastes A of Examples 1-12

| | | | | | FINE | COARSE | | Micro | Zr—Si |
|---|---|---|---|---|---|---|---|---|---|
| Ex. | HEMA | DMAPE | ATU | WATER | GLASS | GLASS | R812S | TiO2 | Filler |
| 1 | 9.38 | 0.38 | 0.38 | 15.63 | 36.27 | 36.27 | 1.2 | 0.5 | 0 |
| 2 | 7.962 | 0.38 | 0.38 | 13.568 | 40.54 | 32 | 1.2 | 0.5 | 3.47 |
| 3 | 7.962 | 0.38 | 0.38 | 13.568 | 38.01 | 38.01 | 1.2 | 0.5 | 0 |
| 4 | 7.962 | 0.38 | 0.38 | 13.568 | 49.01 | 27 | 1.2 | 0.5 | 0 |
| 5 | 7.962 | 0.38 | 0.38 | 13.568 | 33.7 | 42.31 | 1.2 | 0.5 | 0 |
| 6 | 7.962 | 0.38 | 0.38 | 13.568 | 42.31 | 33.7 | 1.2 | 0.5 | 0 |
| 7 | 7.962 | 0.38 | 0.38 | 13.568 | 27 | 49.01 | 1.2 | 0.5 | 0 |
| 8 | 9.38 | 0.38 | 0.38 | 15.63 | 36.27 | 36.27 | 1.2 | 0.5 | 0 |
| 9 | 9.38 | 0.38 | 0.38 | 15.63 | 72.54 | | 1.2 | 0.5 | 0 |
| 10 | 9.38 | 0.38 | 0.38 | 15.63 | 54.405 | 18.135 | 1.2 | 0.5 | 0 |
| 11 | 9.38 | 0.38 | 0.38 | 15.63 | 18.135 | 54.405 | 1.2 | 0.5 | 0 |
| 12 | 9.38 | 0.38 | 0.38 | 15.63 | 0 | 72.54 | 1.2 | 0.5 | 0 |

TABLE 2
Viscosities of Pastes A of Examples 1-12

| Example | Viscosity (Pa*s) |
|---|---|
| 1 | 6.0 |
| 2 | 23 |
| 3 | 16 |
| 4 | 28 |
| 5 | 12 |
| 6 | 19 |
| 7 | 12 |
| 8 | 6.6 |
| 9 | 27 |
| 10 | 14 |
| 11 | 3.8 |
| 12 | 2.9 |

### Comparative Example C13 and Examples 14-22

Pastes B were prepared as described above, and the resulting paste compositions are shown in Table 3. The viscosity of each of the resulting paste compositions was measured according to Test Method IV, and the results are shown in Table 4.

TABLE 3
Compositions of Pastes B of Comparative Example C13 and Examples 14-22

| | | | | | | | | | Zr—Si |
|---|---|---|---|---|---|---|---|---|---|
| Ex. | HEMA | GDMA | R812S | VBCP | BisGMA | BHT | SW | PP | Filler |
| C13 | 15.2 | 0 | 0.21 | 33.78 | 2.89 | 0.128 | 12.08 | 1.94 | 33.78 |
| 14 | 18 | 7 | 0.2 | 23.98 | 0 | 0.128 | 11.35 | 1.94 | 37.4 |
| 15 | 16 | 7 | 0.2 | 23.98 | 0 | 0.128 | 10.75 | 1.94 | 40 |
| 16 | 14 | 9 | 0.2 | 23.98 | 0 | 0.128 | 10.75 | 1.94 | 40 |
| 17 | 16 | 9 | 0.2 | 23.98 | 0 | 0.128 | 11.35 | 1.94 | 37.4 |
| 18 | 32 | 7 | 0.2 | 10 | 0 | 0.128 | 11.35 | 1.94 | 37.4 |
| 19 | 27 | 7 | 0.2 | 15 | 0 | 0.128 | 11.35 | 1.94 | 37.4 |
| 20 | 22 | 7 | 0.2 | 20 | 0 | 0.128 | 11.35 | 1.94 | 37.4 |
| 21 | 20.09 | 5 | 0.2 | 23.89 | 0 | 0.128 | 11.35 | 1.94 | 37.4 |
| 22 | 18.149 | 0 | 0.2 | 28.846 | 0 | 0.116 | 10.95 | 1.9 | 39.84 |

TABLE 4
Viscosities of Pastes B of Comparative
Example C13 and Examples 14-22

| Ex. | Viscosity (Pa*s) |
|---|---|
| C13 | 149 |
| 14 | 22, 25 |
| 15 | NT |
| 16 | NT |
| 17 | NT |
| 18 | 0.3 |
| 19 | 1.6 |
| 20 | 6.5 |
| 21 | NT |
| 22 | 101 |

NT = not tested

### Examples 23-35, 37-42, and Comparative Example C36

Pastes A of Examples 1-12 and pastes B of Examples 14-22 and Comparative Example C13 were auto mixed using the dispensing equipment described above, and the extrusion force was determined according to Test Method I described above. In addition, diametral tensile strength was determined on the auto mixed compositions according to Test Method III described above. The pastes of Examples 1 and C13 and 2 and 21 were also mixed by hand to form hand-mixed Comparative Example C36 and Example 37, respectively. The DTS of these mixed compositions were also determined. The results arc shown in Table 5. In addition, the shear bond strength of the compositions formed by mixing the pastes of Examples 1 and C13 (by hand mixing) and 2 and 21 (by auto mixing) were determined according to Test Method II described above, and the results are shown in Table 6.

TABLE 5
Extrusion Force and Diametral Tensile Strength (DTS) of
Examples 23-35 and 37-42 and Comparative Example C36

| | | | Extrusion | | | | |
|---|---|---|---|---|---|---|---|
| | Paste A | Paste B | Force (lbf = | | | | |
| Exam- | Exam- | Exam- | pounds | | | | DTS* |
| ple | ple | ple | force) | | DTS (MPa) | | (MPa) |
| 23 | 2 | 14 | 14.2 | (0.078) | 23.8 | (2.49) | NT |
| 24 | 2 | 15 | 18.05 | (0.25) | 24.1 | (2.7) | NT |
| 25 | 2 | 17 | 14.92 | (0.3) | 24.9 | (0.83) | NT |
| 26 | 2 | 16 | 20.3 | (1.3) | 25.3 | (1.3) | NT |
| 27 | 2 | 14 | 12.8 | | 24.3 | (1.6) | NT |
| 28 | 2 | 18 | 5.2 | | 15 | (0.5) | NT |
| 29 | 2 | 19 | 6.55 | | 16.7 | (2.3) | NT |
| 30 | 2 | 20 | 8.5 | | 18.5 | (1.8) | NT |
| 31 | 4 | 14 | 14.7 | (0.36) | 22.76 | (1.312) | NT |
| 32 | 6 | 14 | 12.8 | (0.06) | 23.04 | (2.43) | NT |
| 33 | 3 | 14 | 11.9 | (0.52) | 22.03 | (3.17) | NT |
| 34 | 5 | 14 | 11.1 | (0.01) | 21.05 | (2.45) | NT |
| 35 | 7 | 14 | 10.7 | (0.51) | 21.17 | (2.68) | NT |
| C36 | 1 | C13 | 40.2 | | 10.3 | (1.5) | 21.6 |
| | | | | | | | (2.36) |
| 37 | 2 | 21 | 13 | | 24.3 | (1.6) | 24.2 |
| | | | | | | | (1.2) |
| 38 | 8 | 22 | 26.2 | (0.2) | 23.90 | (3) | NT |
| 39 | 9 | 22 | 31.7 | (1.1) | 21.61 | (2.43) | NT |
| 40 | 10 | 22 | 26.3 | (0.49) | 25.92 | (2.28) | NT |
| 41 | 11 | 22 | 27.2 | (0.98) | 19.55 | (2.32) | NT |
| 42 | 12 | 22 | 25.7 | (1.10) | 15.7 | (5.78) | NT |

*This is the DTS resulting after hand mixing by spatulation
( ) Numbers in parenthesis are standard deviation values.

It can be seen in Table 5, that Comparative Example C36 required a much higher extrusion force for auto mixing that the other Examples. In addition, the DTS of C36 was considerably lower when auto mixed compared with hand mixing, illustrating an effect of inadequate mixing, at least in part resulting from a mismatch in viscosities of paste A of Example 1 and paste B of Comparative Example C13.

TABLE 6
Shear Bond Strengths of Example 37 and Comparative Example C36

| | | | Shear | Shear | Shear | Shear |
|---|---|---|---|---|---|---|
| | | | Bond | Bond | Bond | Bond |
| | Paste A | Paste B | Strength | Strength* | Strength | Strength* |
| Exam- | Exam- | Exam- | (Dentin) | (Dentin) | (Enamel) | (Enamel) |
| ple | ple | ple | (MPa) | (MPa) | (MPa) | (MPa) |
| C36 | 1 | C13 | NT | 10.6 | NT | 8.17 |
| | | | | (3.21) | | (1.21) |
| 37 | 2 | 21 | 7.4 | NT | 6.6 | NT |
| | | | (2.21) | | (0.89) | |

*This is the shear bond strength of the composition resulting after hand mixing by spatulation.
( ) Numbers in parenthesis are standard deviation values.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

The complete disclosures of the patents, patent documents, and publications cited herein are incorporated by reference in their entirety or the portions of each that are indicated as if each were individually incorporated.

1. A method of dispensing a hardenable dental composition comprising:
providing a multi-part hardenable dental composition comprising:
a part (A) in the form of a paste, comprising:
acid-reactive glass particles and a liquid selected from the group consisting of water, a monomer having at least one ethylenically unsaturated group per monomer molecule, and a combination thereof; and
a part (B) comprising:
a water miscible polyacid and a liquid selected from the group consisting of water, a monomer having at least one ethylenically unsaturated group per monomer molecule, and a combination thereof;
wherein:
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B); and
extruding the composition through a static mixer in fluid communication with a first reservoir containing the part (A) and a second reservoir containing the part (B);
wherein a plunger is positioned in each reservoir for simultaneously forcing part (A) and part (B) into the static mixer, extruding the composition through the static mixer, and dispensing the composition; and
wherein an extrusion force of less than 40 pound-force (178 newtons) according to Test Method I is applied to the plunger for extruding the composition through the static mixer without the aid of a mechanical advantage provided by an attached or external device.

2. A method of bonding a prosthetic device to a dental structure comprising:
dispensing the hardenable dental composition according to claim 1 onto a surface of a dental prosthetic device, a surface of a dental structure, or a combination thereof;
positioning the device on the dental structure; and
hardening the dental composition;
wherein the prosthetic device is selected from the group consisting of a crown, bridge, inlay, onlay, post, abutment, veneer, and prosthetic tooth; and
wherein the dental structure is a prepared tooth or an implant.

3. A dental device comprising:
a multi-part hardenable dental composition comprising:
a part (A) in the form of a paste, comprising:
acid-reactive glass particles and a liquid selected from the group consisting of water, a monomer having at least one ethylenically unsaturated group per monomer molecule, and a combination thereof; and
a part (B) comprising:
a water miscible polyacid and a liquid selected from the group consisting of water, a monomer having at least one ethylenically unsaturated group per monomer molecule, and a combination thereof;
wherein:
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B);
a first reservoir containing the part (A);
a second reservoir containing the part (B);
a static mixer in fluid communication with or which can be connected in fluid communication with the first and second reservoirs; and
a plunger positioned in each reservoir for forcing part (A) and part (B) into the static mixer, extruding the composition through the static mixer, and dispensing the composition;
wherein an extrusion force of less than 40 pound-force (178 newtons) according to Test Method I is required for extruding the composition through the static mixer without the aid of an attached or external device for providing a mechanical advantage.

4. A dental kit comprising the device of claim 3 and a plurality of static mixers adapted for fluid communication with the first and second reservoirs.

5. A multi-part hardenable dental composition comprising:
a part (A) in the form of a paste, comprising:
acid-reactive glass particles and a liquid selected from the group consisting of water, a monomer having at least one ethylenically unsaturated group per monomer molecule, and a combination thereof; and
a part (B) comprising:
a water miscible polyacid and a liquid selected from the group consisting of water, a monomer having at least one ethylenically unsaturated group per monomer molecule, and a combination thereof;
wherein:
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B);
wherein the composition can be extruded through a static mixer in fluid communication with a first reservoir containing the part (A) and a second reservoir containing the part (B);
wherein a plunger is positioned in each reservoir for simultaneously forcing part (A) and part (B) into the static mixer and extruding the composition through the static mixer; and
wherein an extrusion force of less than 40 pound-force (178 newtons) according to Test Method I is applied to the plunger for extruding the composition through the static mixer without the aid of a mechanical advantage provided by an attached or external device.

6. The method of claim 1, wherein the static mixer includes at least 8 mixing elements.

7. The method of claim 1, wherein when part (A) is mixed with part (B) and the mixture hardened, Shear Bond Strength according to Test Method II of the resulting hardened cement is greater than 2.0 MPa.

8. The method of claim 1, wherein:
part A comprises:
the acid-reactive glass particles, and
a water soluble liquid monomer having one ethylenically unsaturated group per monomer molecule; and
part B comprises:
the polyacid;
a water soluble liquid monomer having one ethylenically unsaturated group per monomer molecule; and
water.

9. The method of claim 8, the device of claim 8, the kit of claim 8, or the composition of claim 8, wherein part B further comprises a liquid monomer having at least two ethylenically unsaturated groups per monomer molecule and having a viscosity less than or equal to the viscosity of bisphenol A diglycidyl methacrylate.

10. The method of claim 1, wherein part (A) and part (B) each independently have a viscosity not less than 6 pascal-second (Pa·s) and not greater than 100 Pa·s; and wherein the ratio of part (B) to part (A) viscosity is 1:0.06 to 1:13.

11. The method of claim 10, wherein the ratio of part (B) to part (A) viscosity is 1:0.6 to 1:3.5.

12. The method of claim 1, wherein the acid-reactive glass particles are a mixture of coarse particles and fine particles, wherein the fine particles have an average particle diameter of about 0.2 to about 2 micrometers, the coarse particles have an average particle diameter of greater than about 2 to about 30 micrometers, and the weight ratio of fine to coarse is 1:3 to 3:1.

13. The method of claim 1, wherein the acid-reactive glass particles are present in part (A) in an amount of 65 to 80 weight percent.

14. The method of claim 1, wherein part (A) includes water.

15. The method of claim 14, wherein the amount of water in part (A) is 7 to 15 percent by weight based upon the total weight of part (A).

16. The method of claim 1, wherein part (B) includes water in an amount of 7 to 15 percent by weight based upon the total weight of part (B).

17. The method of claim 1, wherein part (A), part (B), or part (A) and part (B) further include a nonreactive filler in an amount of 1 to 40 weight percent based upon the total weight of the part which includes the nonreactive filler.

18. The method of claim 17, device of claim 17, the kit of claim 17, or the composition of claim 17, wherein the nonreactive filler is selected from the group consisting of inorganic material, crosslinked organic material, and a combination thereof.

19. The method of claim 17, wherein part (B) includes the nonreactive filler in an amount of 30 to 40 weight percent based upon the total weight of part (B).

20. The method of claim 19, wherein the nonreactive filler is selected from the group consisting of fumed silica, zirconia-silica, quartz, nonpyrogenic silica, and a combination thereof.

21. The method of claim 1, wherein part (B) is in the form of a paste.

22. The method of claim 8, wherein the water soluble liquid monomer is selected from the group consisting of 2-hydroxyethyl methacrylate, glycerol mono(meth)acrylate, sugar methacrylates, and a combination thereof.

23. The method of claim 9, wherein the monomer having at least two ethylenically unsaturated groups per monomer molecule is water insoluble.

24. The method of claim 23, wherein the water insoluble monomer is glycerol dimethacrylate.

25. The method of claim 1, wherein the polyacid is of the formula: B(X)ₘ(Y)ₙ
wherein B is a hydrocarbon backbone, X is —COOH, Y is an ethylenically unsaturated group, m is at least 2, n is at least 1, and Y is attached to B via an amide linkage.

26. The method of claim 25, wherein the polyacid is selected from the group consisting of the reaction product of a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof with a coupling compound selected from the group consisting of acryloyl chloride, methacryloyl chloride, vinyl azalactone, allyl isocyanate, 2-hydroxyethyl methacrylate, 2-aminoethylmethacrylate, and 2-isocyanatoethyl methacrylate.

27. The method of claim 1, wherein the acid-reactive glass is FAS glass.

28. The method of claim 1, wherein the part (A) and part (B) are in a volume ratio of 1.2:1 to 1:1.2.

29. The method of claim 1, wherein the force is less than 30 pound-force (133 newtons).

30. The method of claim 1, wherein the multi-part hardenable dental composition can undergo hardening by photopolymerization, redox polymerization or both.

31. The method of claim 1, wherein the multi-part hardenable dental composition is selected from the group consisting of a liner material, a luting material, a restorative material, an endodontic material, and a sealing material.

32. The method of claim 1, wherein the multi-part hardenable dental composition is an orthodontic bracket adhesive material or band cement.
